(19) United States
(12) Patent Application Publication
(10) Pub. No.: US 2023/0021780 A1
(43) Pub. Date: Jan. 26, 2023

(54) Coronary Therapeutics Decision Aid

(71) Applicant: Robert Francis Riley, Terrace Park, OH (US)

(72) Inventor: Robert Francis Riley, Terrace Park, OH (US)

(21) Appl. No.: 17/443,096

(22) Filed: Jul. 20, 2021

(51) Int. Cl. G16H 20/40; A61B 6/00; G16H 50/20; G16H 10/60

(52) U.S. Cl. CPC G16H 20/40; A61B 6/504; G16H 10/60; G16H 50/20

(57) ABSTRACT

An invention is described herein in which the current subjective decisions to perform a percutaneous coronary intervention (PCI, i.e., “coronary stenting”) for coronary artery disease (CAD) is replaced with consistent and objective algorithm-driven decision trees based on the results of recent clinical trials and studies and objective analysis of the coronary angiogram. This should result in reduced risk and medical cost for patients.

physician’s interpretation of the severity of the blockages seen during the angiogram. Unfortunately, the insurance company has to decide about whether to cover a PCI prior to the diagnostic angiogram being performed given that most PCI are performed “ad hoc”, meaning in the same setting as the diagnostic angiogram. However, the performing physician's interpretation of the clinical data and angiogram is significantly prone to bias. This subconscious (and at times conscious) bias most likely includes interviewer bias, where symptoms and clinical information may be retrospectively amplified to fit the results of the angiogram in order to support a decision to move forward with PCI at the time of the angiogram, and confirmation bias, where providers look for information or patterns in their data that confirm the ideas or opinions that they already hold in order to proceed with PCI.

As can be seen from the above description, there is an ongoing need for a consistent and objective method to make treatment determinations on the appropriateness of PCI versus treatment with medications for CAD after a diagnostic angiogram has been performed based on the results of up-to-date clinical data. Such a method can beneficially reduce medical costs and patient risk by using medications where appropriate instead of PCI (i.e., “receiving a stent”).

### BRIEF DESCRIPTION OF THE FIGURES

The invention can be better understood with reference to the following drawings and description

FIG. 1 represents the coronary therapeutics decision aid process at the core of the invention.

FIG. 2 represents the decision tree for patients with stable CAD not involving the left main coronary artery.

FIG. 3 represents the decision tree for patients with stable CAD involving the left main coronary artery. When CAD involves the left main coronary artery, the risk profile for the PCI procedure is different and current medical literature data points to a different approach compared to CAD that does not involve the left main coronary artery.

FIG. 4 represents the decision tree for patients with unstable angina (UA) or a non-ST segment elevation myocardial infarction (NSTEMI). Heart attacks can come in three forms: UA, NSTEMI, and STEMI. These three forms have different presentations and clinical data around how to treat the blockage(s) causing them. The data for treating UA and NSTEMI is similar, which is why they are lumped into the same decision tree.

FIG. 5 represents the decision tree for patients presenting with a ST-segment elevation myocardial infarction (STEMI).

### DETAILED DESCRIPTION

The decision aid process uses data inputs that are readily obtained from a patient's medical records and the results of quantitative coronary angiography (QCA), which is an objective method of quantifying the degree of stenosis (i.e., “degree of blockage”) in a coronary vessel using software that allows for precise measurement of the blockage. This is a much more precise way of measuring the degree of blockage compared to the current standard method where the treating physician “eyeballs” the angiogram at the time of the procedure and comes up with a subjective measure of stenosis . These data inputs are then processed through the above referenced decision algorithms that outputs one of four potential treatment options: optimal medical therapy (i.e., treatment with medications), PCI, a stress test, or a heart team approach to revascularization, where a team of physicians decide on whether to perform PCI or CABG surgery for complex CAD (these algorithms do not attempt to guide this particular decision).

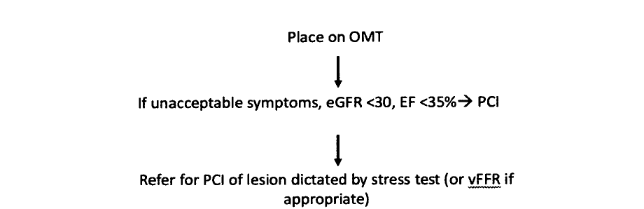

Aside from the degree of stenosis obtained by QCA, the other data inputs are: current medications, clinical presentation (stable CAD, STEMI, or UA/NSTEMI), current symptoms (based on the objective Canadian Classification System [CCS] classification of chest pain and the New York Heart Association [NYHA] classification of shortness of breath, both of which are symptoms of CAD), and degree of vessel stenosis. The degree of vessel stenosis is determined by a trained physician using computer software to assign a numerical value representative of the degree of vessel stenosis (blockage)- this method is called quantitative coronary analysis (QCA). These inputs are all either binary (yes/no to current medications), categorical (presentation), ordinal (current symptom classification), or continuous (degree of stenosis). The inputs except for angiogram analysis are uploaded to a HIPPA-compliant cloud storage system. The coronary angiogram is also uploaded to the same storage system, whereby it can undergo QCA. These inputs are then put into the decision tree algorithms (FIGS. 2-5) and a decision is made on whether to proceed with a PCI procedure or not. This output is then given back to the treating physician.

We have also developed an algorithm utilizing machine learning that will further refine quantitative vessel stenosis measurements beyond the currently available methods in QCA. This algorithm will both a) continue to evolve in further refining quantitative vessel stenosis measurements as further data (angiograms) are integrated into our data set once in use in a larger population). This algorithm was developed with the open-source Cleveland Clinic Heart Disease Dataset (https://www.kaggle.com/aavigan/cleveland-clinic-heart-disease-dataset). As the algorithm is put into practice on a larger scale, it will continue to learn (initially with human input on evaluating vessel stenosis by QCA) and ultimately be able to perform QCA to a more precise degree than human measurements while also adjusting for any bias found in the model. The algorithm is an ensemble (i.e. “hurstruistic”) model, which takes each of the inputs and creates and evaluates multitude models in order to determine the most precise model. It was determined that gradient boosting classifier was chosen as the best fit for the data we used to develop the model. This was considered a “base” model. With this model, we were able to generate outcomes that correlated with 88% of outcomes generated by clinicians using the same inputs. The power of an ensemble strategy will allow this model to continue to refine itself as human inputs for the outcomes are further integrated into the model in order to continue to improve the correlation with human predictions.

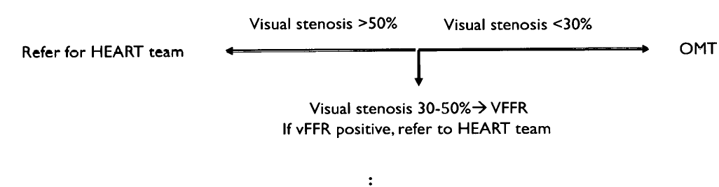

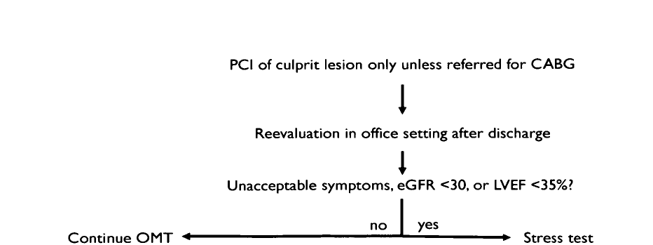

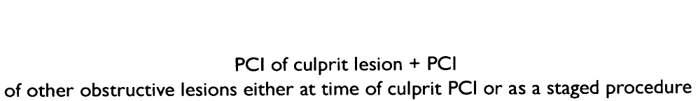

The treatment decision algorithms are generally represented in FIGS. 2 through 5. The threshold as to which decision algorithm to use is based on the patient’s clinical presentation-whether they have stable CAD, a STEMI, or UA/NSTEMI. In the instance of stable CAD, the steps described of FIGS. 2 and 3 are applied. In the instance of UA/NSTEMI, the method of FIG. 4 is applied. In the instance of STEMI, the method of FIG. 5 is applied.

FIG. 2 represents a stable CAD decision tree where the angiogram shows non-left main CAD. After the angiogram is obtained, the procedure is halted. The decision tree recommends that the patient is placed on optimal medical treatment (OMT) and the patient is reevaluated in an office setting. If unacceptable symptoms (CCS class III-IV angina or NYHA class III-IV dyspnea are believed due to coronary ischemia), then the patient is referred for a stress test to guide revascularization and then referred for PCI of lesion dictated by the stress test. If significant LV dysfunction (LVEF <35%) is present, patients with single vessel CAD would undergo PCI and those with multivessel CAD involving the proximal LAD would prompt a heart team multidisciplinary discussion on revascularization (PCI versus CABG surgery).

FIG. 3 represent a stable CAD decision tree for patients with left main CAD where the coronary angiogram shows a lesion in the left main coronary artery. If the visual stenosis input is > 50%, then the algorithm recommends a heart team approach to revascularization (PCI versus CABG surgery). If the visual stenosis input is < 30%, then the decision algorithm recommends optimal medical therapy. If the visual stenosis input is 30% to 50%, the decision algorithm recommends an invasive stress test (fractional flow reserve [FFR]) or intravascular imaging to better determine the significance of the lesion, unless the patient has already had a positive stress test. If the FFR or intravascular imaging test indicates significant left main CAD, then the decision algorithm provides a recommendation a heart team multidisciplinary discussion on revascularization (PCI versus CABG surgery). If the FFR or imaging is not suggestive of significant left main CAD, then the decision algorithm provides a recommendation of optimal medical therapy.

FIG. 4 represents a decision tree for patients with UA or NSTEMI presentation, where an angiography is followed by revascularization for the culprit lesion only plus optimal medical therapy unless the patient’s blockages dictate referral for heart team multidisciplinary discussion on revascularization (PCI versus CABG surgery). If the patient undergoes PCI of the culprit stenosis during the hospital stay, the patient is then reevaluated in an office setting after discharge on optimal medical therapy. If unacceptable symptoms persist (CCS class III angina or NYHA class III OMT) persist or here is significant LV dysfunction (LVEF <35%), then the patient is referred for a stress test to determine which lesions to revascularize. Otherwise, the optimal medical therapy is continued.

FIG. 5 represents a STEMI decision tree where an angiography is followed by revascularization for the culprit lesion (the lesion causing the heart attack) plus revascularization of all non-culprit lesions (stenosis > 90% in a 2 mm or greater sized vessel or positive FFR measurement for lesions visually 50% to 90% in stenosis) either at the time of the culprit PCI or in a separate, staged procedure.

After the input data is applied to the appropriate decision tree, the treatment decision algorithm provides a result in the form of one of four possible treatment plans. In general, the four possible treatment plan results provided by the decision algorithm are initiating or continuing optimal medical therapy, a stress test with physician follow-up, PCI, or a heart team approach to revascularization.

The following examples illustrate one or more embodiments of the invention. Numerous variations may be made to the following examples that lie within the scope of the invention.

### EXAMPLES

Example 1: Medication Therapy Recommended by Treatment Decision Algorithm for Stable CAD Presentation.

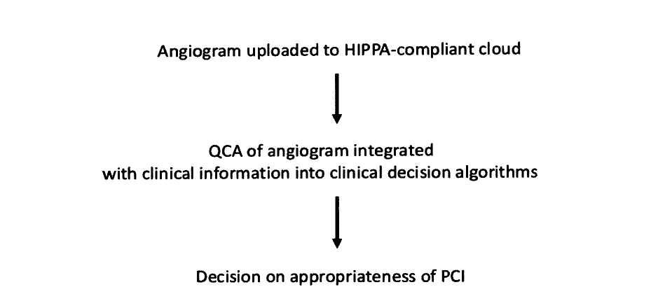

Mr. T is a 56-year-old male that developed chest pain. He is placed on one anti-chest pain medication and then sent for a coronary angiogram. The clinical variables and angiogram are then uploaded to the HIPPA-compliant cloud, which allows for independent analysis (FIG. 1). QCA is performed on the angiogram and degree of coronary artery stenosis (es) are determined. In this case example, QCA shows two vessels with significant blockages (>/= 70%) but did not involve the left main coronary artery. The coronary stenosis value along with the pertinent clinical information is processed through the treatment decision algorithm shown in FIG. 2, which provides the result of trying an additional medication instead of PCI at that time. The patient is then sent back to his regular cardiologist with the recommendation of trying a second anti-chest pain medication, as provided by the algorithm. His regular cardiologist places him on a second anti-chest pain medication, which takes his chest pain to a much less significant level (CCS class II), and he is able to do most things in life without significant symptoms. The angiogram value along with the pertinent clinical information is then reprocessed through the treatment decision algorithm, which provides the result of continuing medication to treat the blockages instead of PCI.

Example 2: Stent Therapy Recommended by Treatment Decision Algorithm for Stable CAD Presentation.

Ms. S is a 74-year-old female that develops chest pain. She is placed on one anti-chest pain drug and then sent for coronary angiography. The clinical variables and angiogram are then uploaded to the HIPPA-compliant cloud, which allows for independent analysis (FIG. 1). QCA is performed on the angiogram and degree of coronary artery stenosis (es) are determined. In this case example, QCA shows one vessel with a significant blockage (>/= 70%). The angiogram value along with the pertinent clinical information is processed through the treatment decision algorithm as shown in FIG. 2, which provides the result of trying an additional anti-chest pain medication instead of PCI. The patient is then sent back to her regular cardiologist who puts her on a second anti-chest pain medication, which unfortunately does not change her symptoms very much and she remains CCS class III. This additional input is then processed through the decision algorithm, which provides a result recommending PCI to fix the blockage instead of continuing the medications previously recommended to treat the blockage.

Example 3: Medication Therapy Recommended by Treatment Decision Algorithm for Unstable Angina/ NSTEMI.

Ms. H is a 66-year-old female who has a small heart attack (NSTEMI). She is admitted to the hospital and undergoes coronary angiography with PCI of the blockage causing her heart attack in the same sitting (i.e., “ad hoc”). However, it is noticed that a blockage exists in a second coronary artery during coronary angiography which did not cause the heart attack but seems significant to the physician treating her. She is then discharged home and follows up with her cardiologist. She reports CCS class III symptoms at that time. She is on one anti-chest pain medication at that time. The clinical variables and angiogram are then uploaded to the HIPPA-compliant cloud, which allows for independent analysis (FIG. 1). QCA is performed on the angiogram and degree of coronary artery stenosis (es) are determined. In this case example, QCA shows that the blockage is 60%. Her inputs are processed through the treatment decision algorithm shown in FIG. 4, which recommends trying a second anti-chest pain medication before proceeding to PCI. After starting the additional medication, she follows up with her cardiologist again, noting at that time that her chest pain is now much less significant (CCS class II) and she is able to most things in life without significant symptoms. Therefore, she continues treating this second, non-stented, blockage with medications instead of a second PCI procedure.

Example 4: Treatment Decision Algorithm for patient with STEMI.

Mr. J is a 65-year-old male who enters the hospital with a large heart attack (STEMI). The treating physician fixes the blockage causing the heart attack but notices there is a significant blockage in another vessel. The patient is discharged and follows up with his regular cardiologist. The clinical variables and angiogram are then uploaded to the HIPPA-compliant cloud, which allows for independent analysis (FIG. 1). QCA is performed on the angiogram and degree of coronary artery stenosis (es) are determined. In this case example, QCA shows that the blockage is 80%. His inputs are processed through the treatment decision algorithm in the FIG. 5 decision tree, which recommends the patient undergo PCI of the remaining Blockage.

To provide a clear and more consistent understanding of the specification and claims of this application, the following definitions are provided below:

PCI stands for percutaneous coronary interventions or coronary stenting

CAD stands for coronary artery disease

CABG stands for coronary artery bypass graft surgery

QCA stands for quantitative coronary analysis

FFR stands for fractional flow reserve, which is a type of invasive stress test

] UA stands for unstable angina

NSTEMI stands for a non-ST elevation myocardial infarction, also known as a “small heart attack”

STEMI stands for ST-elevation myocardial infarction, also known as a “large heart attack”

OMT stands for optimal medical therapy

While various aspects of the invention are described, it will be apparent to those of ordinary skill in the art that other embodiments and implementations are possible within the scope of the invention. Accordingly, the invention is not to be restricted except in light of the attached claims and their equivalents.

### CLAIMS

This invention is a method of determining whether a patient with significant coronary artery disease should undergo PCI, a stress test, drug therapy, or a heart team approach to revascularization based on inputting patient data comprising values for current medications; clinical presentation chosen from stable CAD, STEMI, and UA/NSTEMI; current symptoms, and degree of vessel stenosis by QCA; and processing the values representing the

1. This invention is a method of determining whether a patient with significant coronary artery disease should undergo PCI, a stress test, drug therapy, or a heart team approach to revascularization based on inputting patient data comprising values for current medications; clinical presentation chosen from stable CAD, STEMI, and UA/NSTEMI; current symptoms, and degree of vessel stenosis by QCA; and processing the values representing the patient data through a treatment decision algorithm as represented in Figures 2 through 5, where the treatment decision algorithm recommends a treatment result chosen from continuing drug therapy, additional drug therapy, stress test, PCI, or a heart team approach. These decision tree algorithms also have the ability to incorporate machine learning into further refining the QCA component of the decision trees beyond the current human-driven method of analysis. Finally, these algorithms will be able to incorporate additional data as part of a neural network in further refining important clinical factors that are significant predictors of the measured outcome of recommending medical therapy versus PCI as more data is analyzed by the algorithms. This method can also be extrapolated to other methods of defining coronary and peripheral angiography including but not limited to computed tomography or magnetic resonance angiography (CTA or MRA), incorporating similar information into the decision trees seen in Figures 2-5.
